(19) United States
(12) Patent Application Publication
(10) Pub. No.: US 2005/0288743 A1
(43) Pub. Date: Dec. 29, 2005

(54) Implantable medical device with contactless power transfer housing

(76) Inventors: Tae Young Ahn, Cheongju-si (KR); Byung Joon Kim, Baltimore, MD (US); Moon Chanil, Baltimore, MD (US)

Correspondence Address: LAW OFFICES OF ROYAL W. CRAIG;A PROFESSIONAL CORPORATION, SUITE 153, 10 NORTH CALVERT STREET, BALTIMORE, MD, 21202, US

(21) Appl. No.: 11/143,108

(22) Filed: Jun. 2, 2005

Related U.S. Application Data
(63) Continuation-in-part of application No. 09/949,612, filed on Sep. 12, 2001.

(30) Foreign Application Priority Data
May 23, 2001 (KR) KR 2001-28347

(51) Int. Cl. A61N 1/08

(52) U.S. Cl. 607/61

(57) ABSTRACT

A transcutaneous recharging system for providing power to an implantable medical device comprises a primary side circuit for transmitting power in the form of magnetic flux; and a secondary side circuit integral to the implantable medical device for receiving the power transmitted from the primary side circuit and for providing the received power to recharge a battery in the implantable medical device, wherein the primary and secondary side circuits are not physically coupled. A variety of attachment configurations are disclosed for attaching and shielding the secondary circuit directly onto the housing of the implantable medical device, inclusive of flexible printed circuit coils and wire coils recessed into helical notches.

### CROSS-REFERENCE TO RELATED APPLICATIONS

The present application is a continuation-in-part of U.S. Application Serial No. 949612, filed Sep. 12, 2001, which in turn derives priority from Korean Application Serial No. KR 2001-28347 filed May 23, 2001. The aforesaid applications are commonly owned by the named inventors.

### BACKGROUND OF THE INVENTION

1. Field of the Invention

The present invention relates to implantable medical devices such as pacemakers and defibrillators and, more particularly, to an improved rechargeable power supply configuration including a remote primary circuit for contactless charging, and a housing design for the implantable medical device that incorporates a non-contact secondary circuit for charging by the remote primary circuit.

2. Description of the Background

It is forecast that the US market for implantable medical devices will grow 10.9% per year through 2007, to nearly $24.4 billion. The growth leaders are anticipated to be cardiac resynchronization devices, implantable cardioverter defibrillators (ICDs), drugeluting stents, bioengineered tissue implants, neurological stimulators, cochlear implants and retinal implants. Much of this growth is due to technological advances in the devices themselves which make them less obtrusive and more reliable. Also, based on increasing clinical evidence of therapeutic effectiveness and lifesaving benefits, third-party insurance concerns are covering an expanding number of heart patients for pacemakers, implantable cardioverter defibrillators and coronary stents. These devices are enabling persons afflicted with cardiac rhythm disorders and heart failure to live a more normal life without dependence on complex drug regimens. The most pressing need for further technological advances lies in the size and weight of implanted devices, and this remains the major challenge for many researchers. The size of an implanted device directly affects the comfort of the patient. Particularly, if an implant is large it will require that much large opening in the living body either to insert or remove it, possibly causing an excessive bleeding and increasing vulnerability to infection during the implantation.

A battery occupies 50 to 80% of volume in most of implanted medical devices. However, batteries have a limited lifespan and must be replaced periodically. The replacement also requires a surgical operation to make an opening in the body, which is very inconvenient to and can be dangerous for some patients. For this reason, transcutaneous power transmission has been tired as a form of non-contact power transmission.

For example, a prior art charger for implanted medical device is disclosed in U.S. Pat. No. 4,143,661, which shows a very large coil implanted in a human body so as to surround a leg or the waist to use it as the secondary coil. Implanting such a large coil adversely affects the patient's condition. In addition, a large coil inserted into a human body could cause damages to the body.

Another prior art charger is disclosed in U.S. Pat. No. 5,358,514. The charger disclosed therein includes a secondary transformer, a battery and other supplemental circuitry. For magnetic flux supplied from outside of a human body to reach the charger, the charger cannot be enclosed in a metal case, which imposes restrictions on the design of the implanted device. Since ferromagnetic core surrounded by a coil is used as a component of a secondary transformer, it is bulky and vulnerable to impact from outside.

Yet another prior art charger is disclosed in U.S. Pat. No. 6,505,077 to Kast et al., which shows a recharging coil 54 carried on the housing exterior surface 64 of a medical device 20. The recharging coil 54 is manufactured from copper wire, copper magnet wire, copper litz woven wire, gold alloy and the like, and is coupled to recharging feedthroughs 68 with an electrical connection 56.

None of the foregoing nor any known contactless battery charging systems are well-adapted for incorporation directly in/on the housings of existing implantable medical devices, rather than at remote locations. This is because existing designs are too bulky and unsuitable for implantation, are too prone to oxidation once implanted (and to poisoning the patient), are too inefficient for practical charging, or are simply incompatible with the materials of most implantable medical devices. For example, for magnetic flux supplied from outside of a human body to reach a charger, the charger cannot be enclosed in a metal case.

Consequently, it would be greatly advantageous to provide a completely sealed and safe contactless battery charging system with secondary coils that can be incorporated directly in/on the housings of most existing implantable medical devices, so as to minimize space.

### SUMMARY OF THE INVENTION

It is, therefore, an object of the present invention to provide a transcutaneous power transmission apparatus for use in an implantable medical device.

It is another object to provide a transcutaneous power transmission apparatus for use in an implantable medical device that is small and compact, and can be implanted with the medical device, thereby minimizing surgery and subsequent treatments.

It is another object to provide a transcutaneous power transmission apparatus for use in an implantable medical device that optimizes the transcutaneous magnetic coupling to minimize charging time.

According to the present invention, the above-described and other objects are accomplished by providing an apparatus for providing power to an implantable medical device comprising a primary side circuit for transmitting power in the form of magnetic flux; and a secondary side circuit integral to the implantable medical device for receiving the power transmitted from the primary side circuit and for providing the received power to recharge a battery in the implantable medical device, wherein the primary and secondary side circuits are not physically coupled. A variety of attachment configurations are disclosed for attaching and shielding the secondary circuit directly onto the housing of the implantable medical device, inclusive of flexible printed circuit coils and wire coils recessed into helical notches. The system can be utilized for various implantable medical devices that requires electrical power, such as an artificial heart, a pacemaker, an implantable cardiverter defibrillator, a neurostimulator, a GI stimulator, an implantable drug infusion pump, a bone growth stimulation device, and many other devices. The system improves the power transmission coupling such that sufficient electric power can be transmitted to the medical device repeatedly without having to take the implanted medical device out of the human body. Further, since charging is more efficient and the secondary coils are integral to the implant housing the size of the battery can be reduced, thereby reducing the overall size of the implanted medical device. Moreover, the secondary coil(s) conform to the implant housing and are hermetically sealed to be non-obtrusive, non-corrosive and medically safe.

### BRIEF DESCRIPTION OF THE DRAWINGS

Other objects, features, and advantages of the present invention will become more apparent from the following detailed description of the preferred embodiment and certain modifications thereof when taken together with the accompanying drawings in which:

FIG. 1 is a side cut-away view of the primary recharging unit 4 used in the present invention.

FIG. 2 is a front view of the primary recharging unit 4 as in FIG. 1.

FIG. 3 is a side cut-away view of the contactless power transfer housing 6 used in the present invention.

FIG. 5 illustrates an exemplary circuit schematic that is suitable for the present invention.

FIG. 6 depicts waveforms of the control signals s1, s2, s3, and s4 applied to the circuit of FIG. 6.

FIGS. 7 and 8 (A&B) illustrate the operation of the contactless power transfer system, inclusive of primary recharging unit 4 located outside the human body and contactless power transfer housing 6 which is part and parcel to the implantable medical device implanted inside the human body.

FIGS. 9-12 illustrate alternative configurations of the secondary side coil(s) 36.

FIG. 13 illustrates two alternative form-fitting embodiments of the secondary unit 6.

FIG. 14 illustrates alternative placements of secondary coils 36.

FIGS. 15 and 16 illustrate the leakage flux paths imparted by the present device.

FIG. 17 is a perspective drawing of one embodiment of a shoulder strap 50 designed to be worn to suspend the primary charging unit 4 at the correct position on the body.

FIG. 18 is a perspective drawing of another embodiment which is a vest 60

### DETAILED DESCRIPTION OF THE PREFERRED EMBODIMENTS

The present invention is a contactless power transfer system for an implantable medical device, which includes a primary recharging unit located outside the human body and a contactless power transfer housing forming a portion of the implantable medical device that is implanted inside the human body. A number of embodiments of the present invention will now be described in details with reference to the accompanying drawings.

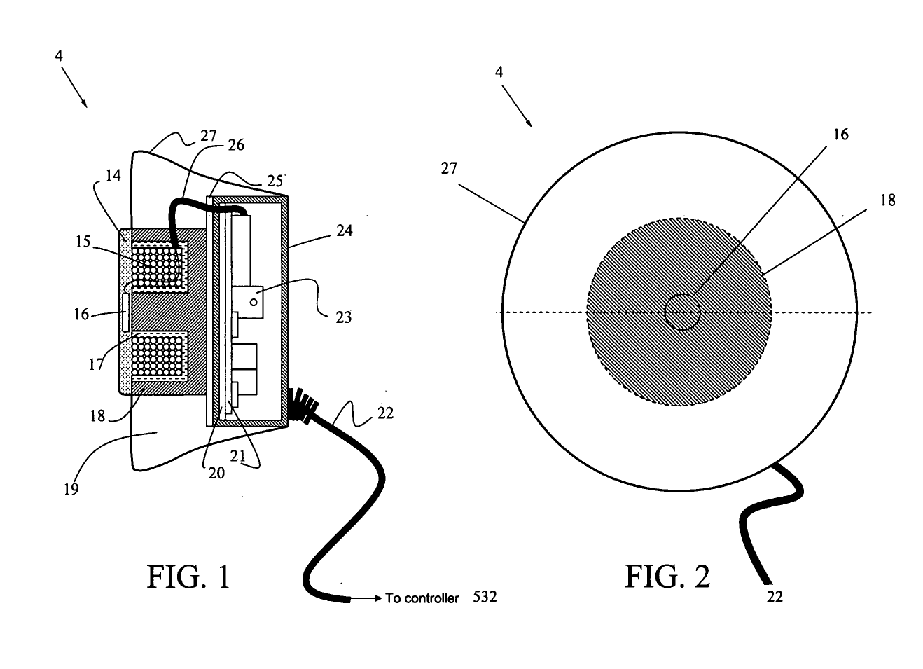

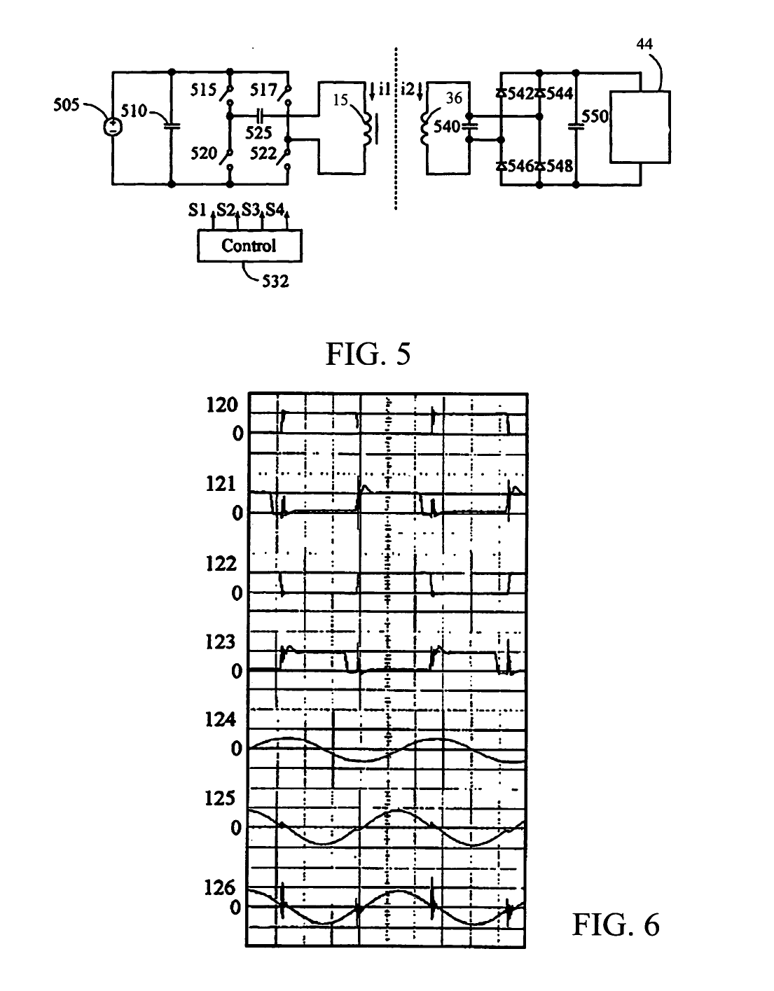

FIG. 1 is a side cut-away view, and FIG. 2 is a front view of the primary recharging unit 4, which generally comprises a toroid-shaped housing 27 with charging coils 15 on one side, and circuit components 23 on the other side that are connectable by power cable 22 to a controller (not shown) for controlled application of recharging power. The controller can be located either inside or outside of the primary recharging unit. The advantage of including the controller inside is minimizing the unit. Furthermore, the primary recharging unit can include some battery unit along with the controller. (All-in-one structure) The housing 27 is preferably filled with an isolation composite 19 such as ferrite, Molypermalloy powder, or Kool Mu®. The recharging power derived from the controller is regulated by the on-board circuit components 23 resident on a printed circuit board 21, and is then applied to the charging coils 15. The circuit components 23 on printed circuit board 21 are contained within an enclosed metal case 24, case 24 being recessed and seated inside housing 27. The charging coils 15 are isolated from the printed circuit board 21 by a layer of isolation material 20, that may be any good electrical insulation material, and which is sandwiched between the circuit board 21 and the back wall of metal case 24. Additionally, the charging coils 15 are isolated from the printed circuit board 21 by a layer of heat insulation material 25, that may be any good heat insulation material, and which is sandwiched between the back wall of metal case 24 and ferrite core 18 (to be described). Charging coils 15 are connected to the circuit components 23 via a power cable 26. The circuit components 23 of printed circuit board 21 generate an AC power transfer signal in a frequency range of from 1-300 kHz. While a variety of circuit designs will suffice for this purpose, FIG. 5 (described later) illustrates one exemplary circuit schematic that is suitable for present purposes. The power transfer signal is transmitted to secondary coils 36 of the medical device that is implanted inside the human body (see FIG. 2 to be described), where it is inductively picked up and converted to a DC recharging signal that is used to charge the battery power source of the implanted medical device.

The charging coils 15 are wound onto a bobbin 17 for stability and ease of assembly, and the bobbin 17 is inserted into a toroid ferrite core 18 that is formed with a circular recess for receiving the bobbin 17. The ferrite core 18 provides EMI shielding capabilities against outside interference and, due to the open-face toroid configuration, directionalizes the transmission to maximize power transmission to the implantable medical device. Ferrite core 18 is preferably an efficient magnetic material such as Alnico (an alloy composed of iron, cobalt, nickel, aluminum, and copper) or Ferrite, but may be may be any other suitable core material such as iron, etc. The primary charging coils 15 are enclosed inside the ferrite core 18 by an isolation composite cover 14, which is a disc of smaller diameter than the toroid-shaped housing 27 and which protrudes slightly beyond the plane of housing 27. The isolation composite cover 14 seals the charging coils 15, bobbin 17 and ferrite core 18 inside the toroid-shaped housing 27, and also positions a flux sensor 16 centrally over the ferrite core 18. Moreover, as seen later the isolation composite cover 14 serves as a skin depressant during use to maximize the magnetic coupling between the primary recharging unit 4 and the secondary. The flux sensor 16 may be a conventional Hall Effect sensor element as used in magnetic field variation meters and the like. The flux sensor 16 may be integrally molded in composite cover 14 such that it is positioned within the air gap of the ferrite core 18, and this is coupled back to the controller to ensure that the correct flux field will be set up within the core 18 material.

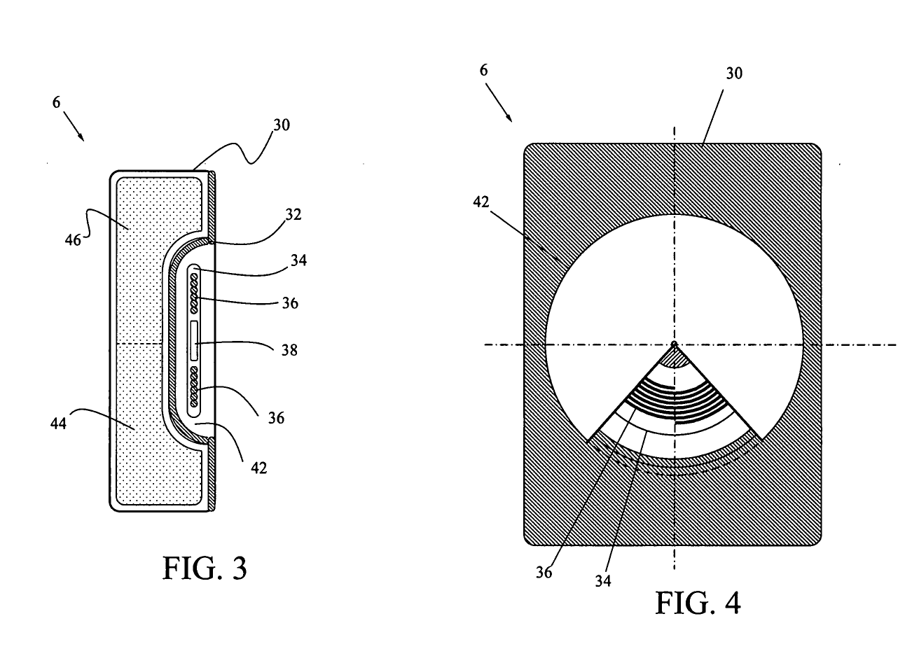

FIG. 3 is a side cut-away view, and FIG. 4 is a front cut-away view of the contactless power transfer housing 6 according to the present invention which forms a portion of an implantable medical device that is implanted inside the human body. The contactless power transfer housing 6 remains integral to the implantable medical device once it has been implanted inside the human body, in contradistinction to prior art contactless charging systems which place secondary coils remotely from the actual implanted device. The contactless power transfer housing 6 generally comprises an enclosed housing 30 formed of conventional implant material such as titanium. The housing 30 contains a rechargeable battery 44 powering any of a variety of implantable medical devices 46, such as an artificial heart, a pacemaker, an implantable cardiverter defibrillator, a neurostimulator, a GI stimulator, an implantable drug infusion pump, a bone growth stimulation device or other electronic devices. It is preferable to use a small and stable battery 44 in the medical device. Lithium-ion and lithium-polymer batteries are examples of small and thin batteries. Although the lithium-ion battery is more efficient, the lithium-polymer battery is preferable because it is more stable.

The front surface of the housing 30 is defined by a circular recess that is covered by a ferromagnetic composite sheet 32 for protection. Sheet 32 may be any thin ferromagnetic sheet material to prevent magnetic flux generated from nearby electronic devices from affecting the medical device, such as a polymer or resin sheet containing iron particles, which may be laminated or coated onto the entire front surface of the housing 30 and across the circular recess. Ferrite compounds in liquid phase, film shape, or solid phase can be utilized as the shield layer 32. The ferrite compounds in liquid phase include a shielding paint that is a mixture of paint and ferrite powder for absorbing electromagnetic flux, such as SMF series products that are produced by Samhwa Electrics. Film type ferrite material includes ferrite polymer compound film supplied by Siemens of Germany. Secondary side coils 36 are contained within the circular recess. When a current is supplied to a coil, magnetic flux is produced in the coaxial direction. Thus, power transmission efficiency is enhanced by placing the flat secondary coil 36 inside the living body oriented directly outward toward the skin such that the primary coils 15 of the recharging unit 4 can be brought into frontal parallel alignment. The secondary side coils 36 are contained within an isolation layer 34. In accordance with the present invention, the secondary side coils 36 are a flat and thin single-layer windings so that they fit flush within the circular recess without disrupting the exterior surface profile of the otherwise small and implantable medical device. A preferred method of forming the secondary side coils 36 integrally with isolation layer 34 is by conventional flex-PCB methods, laminating the coils 36 between opposing polyamide sheets, the plastic then serving as isolation layer 34. Alternatively, the coils 36 may be electronically printed directly onto a polymer substrate, and preferably sealed therein by overlaying a second polymer sheet. A “Flexible PCB” is a term of art in the electronics industry, meaning flexible polyamide film with conductive traces thereon. Flexible printed circuits are thin, lightweight, flexible, durable, and meet a wide range of temperature and environmental extremes such as those encountered in the human body. Flexible printed circuits are well-suited for applications requiring fine line traces (such as coils), and are much better suited for dynamic applications such as human implantation. Moreover, flex PCBs flex and can conform to the exterior housing of most implantable medical devices, taking no additional space. The ability to layer a flexible PCB coil 36 into a recess on the housing 30 greatly reduces manufacturing costs, and the flush configuration also reduces the incision needed to implant the system and avoids complications. Most importantly, the flat concentric coil-to-coil inductive coupling that results gives an efficient transcutaneous power transfer. However, one skilled in the art should understand that the present invention is not confined to flex-PCB methods, as other method exist (and will be described) for arranging a substantially flat single-layer coil 36 onto the surface of an implanted medical device.

The gauge, number of turns, and length of single-layer coil 36 will depend on factors such as desired power transmission, distance from the primary coil outside the living body and battery charging time and may be determined empirically.

A flux sensor 38 is positioned within the air gap of coils 36. As above, the flux sensor 36 may be a conventional Hall Effect sensor element integrally formed in isolation layer 34, and this indicates proper alignment with the Hall Effect sensor 16 on the primary recharging unit 4, which is coupled back to the controller to ensure that the optimum flux field is attained when the primary coils 15 are aligned with secondary coils 36.

The secondary side coils 36, isolation layer 34, and flux sensor 38 are set into a composite material 42 which fills the recess in housing 30 and hermetically seals those components therein. The filler composite 42 is a medically-safe material such silicon or latex which prevents corrosion to the coils 36 and also prevents a possible release of foreign materials from the device inside a living body.

FIG. 5 shows an exemplary circuit schematic of the charging unit 4 and contactless power transfer housing 6 that is suitable for present purposes. In operation, a current is provided to the charging unit 4 from an external power source 505, and switches 515, 517, 520, and 522 are controlled by control signals s1, s2, s3, and s4. The control signals s1, s2, s3, and s4 are generated by the controller of FIG. 1 and correspond to waveforms 120 to 123, respectively, as shown in FIG. 6. When AC current i1 flows in the primary coil 15 by the operation of the switches and a capacitor 525, current i2 is induced in the inductively-coupled secondary coil 36, having substantially the same waveform of current i1. The AC current i2 is rectified to a direct current by diodes 542, 544, 546 and 548. The resultant direct current is provided to charge rechargeable battery 44 of the medical device 6.

FIG. 6 depicts waveforms of the control signals s1, s2, s3, and s4 as well as the currents in the primary and secondary windings 15, 36. Any known circuits for charging a rechargeable battery may be used. Examples of such circuits are MAX745, MAX1679, MAX1736, MAX1879 provided by MAXIM and LTC1732-4/LTC1732-4.2 and LT1571 series provided by Linear technology.

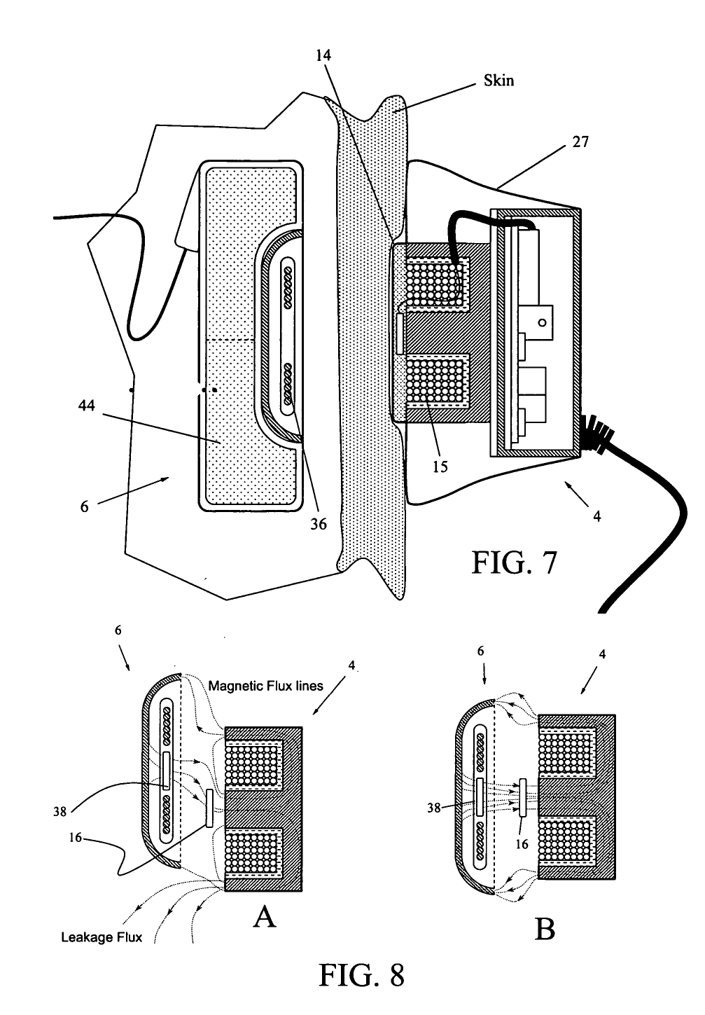

FIGS. 7 and 8 (A&B) illustrate the operation of the contactless power transfer system, inclusive of primary recharging unit 4 located outside the human body and contactless power transfer housing 6 which is part and parcel to the implantable medical device implanted inside the human body. When the internal battery 44 (FIG. 3) is in need of recharging, the noncontact recharging unit 4 is brought into facing proximity to the contactless power transfer housing 6 of the present invention, until as described below with regard to FIG. 8 the flux sensors 16, 36 indicate alignment. By virtue of the isolation composite cover 14 being of smaller diameter and prootruding past the toroid-shaped housing 27, the composite cover 14 serves as a skin depressant as shown, slightly depressing a circular area of skin to maximize the transcutaneous magnetic coupling between the primary recharging unit 4 and the secondary 6.

Power is then applied through the primary recharging unit 4, which delivers the charging signal through the secondary coil 36 to battery 44. The two coils, acting as primary and secondary windings, form a transformer such that power from an external source connected to the primary coil 15 is inductively transferred to the battery 44 coupled to the secondary coil 36.

As seen in FIG. 8A, the primary recharging unit 6 may not initially be perfectly aligned with the contactless power transfer housing 4, especially since the latter is subcutaneous. This is readily apparent from feedback given by flux sensors 38 and 16. With imperfect alignment there will be an uneven flux distribution through the two flux sensors 38, 16. However, as seen in FIG. 8B as the primary recharging unit 6 is better aligned a more even flux distribution occurs through the two flux sensors 16, 38, until the flux distribution is equal. At this point an optimum flux field has been obtained and the primary coils 15 are aligned with secondary coils 36.

One skilled in the art should understand that certain changes may be made without departing from the scope and spirit of the invention. For example, the ferromagnetic composite sheet 32 may cover just the recess at the front of housing 30, but not the entire front of housing 30.

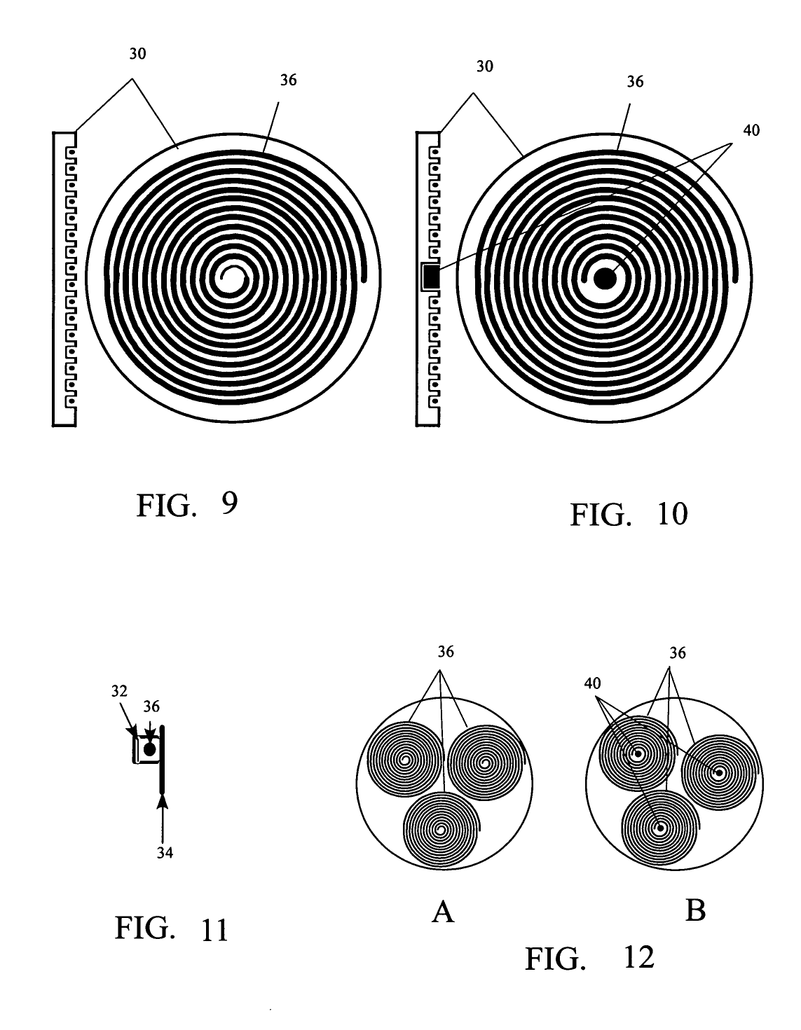

FIGS. 9-12 illustrate alternative configurations of the secondary side coil(s) 36. In FIG. 9, the secondary side coil(s) 36 are formed integrally on the contactless power transfer housing 30 in a coreless configuration. This is accomplished by forming the housing 30 with a helical groove for seating the coil 36. The coil 36 is completely recessed within the groove, and is sealed therein by silicon epoxy or the like.

In this embodiment, the equivalent of the ferromagnetic composite sheet 32 (described in FIG. 3) is implemented by coating a ferrite compound on the housing 30, followed by printing or inlaying the coil windings 36, and then coating the entire outer surface of with a silicon sealant material. It is also possible to eliminate the coating by incorporating ferromagnetic particles in the housing 30 itself, such as by molding the housing 30 with iron particles. Again, the contactless power transfer housing 6 remains integral to the implantable medical device once it has been implanted inside the human body, and in this case the coil 36 is firmly recessed and sealed within the groove. The embodiment of FIG. 10 is similar to that of FIG. 9 except that the secondary side coil(s) 36 are equipped with a core 40 formed integrally in the contactless power transfer housing 30. The core 40 is a simple disc seated centrally in the coil 36 which helps to channel the magnetic flux, thereby ensuring a proper magnetic path and maximum power coupling when transferring power from the primary 4 to the secondary 6. The core 40 should be in contact with the underlying ferromagnetic composite sheet 32 (FIG. 3) or ferromagnetic particles in the housing 30. The material of core 40 may be simple iron, or magnetic materials such as Alnico, Ferrite, etc., which magnetic materials have more efficiency than simple iron.

FIG. 11 is an enlarged drawing illustrating coil 36 completely recessed within the groove, a strip of ferromagnetic composite 32 behind the coil 36 for insulation, and a coating of silicon epoxy 34 sealant over the outer surface.

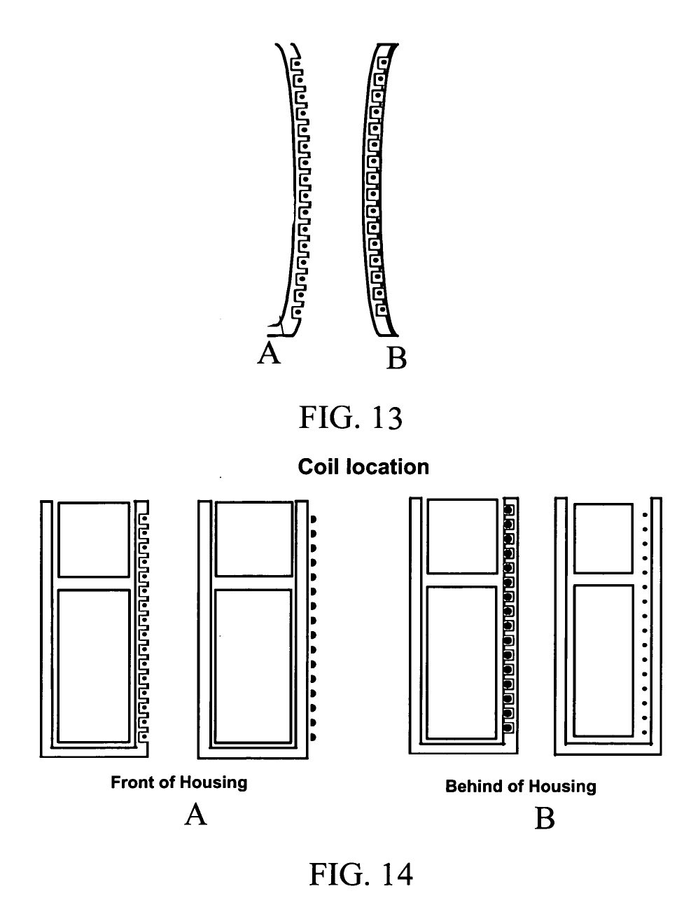

FIG. 12 illustrates a number of alternative multi-coil embodiments in which multiple secondary side coils 36 are formed as adjacent flat and thin single-layer windings, still capable of fitting flush within the housing as described above and not disrupting the exterior surface profile of the otherwise small and implantable medical device. Any number of adjacent secondary side coils 36 may be incorporated as a matter of design choice, three being shown. Each may be equipped with an air core as at (A), or a ferromagnetic core as at (B) to provide a flux path.

FIG. 13 illustrates two form-fitting embodiments similar to that of FIG. 9 but better suited for use with implantable medical devices that do not have a housing with a flat surface. Their surface may be convex or concave. In either case, the secondary coils 36 can be made to conform by forming the housing 30 by seating them in grooves that are graduated so that they conform to the surface profile, such that when the coils 36 are inlayed they are either convex outward (as at A) or convex inward (as at B). By patterning the grooves in housing 30 and laying the coils 36 in the patterned grooves the coils 36 can be made to conform to devices with irregular surfaces. Still, the coil(s) 36 are completely recessed within the groove, and may be sealed therein by silicon epoxy or the like.

FIG. 14 illustrates alternative placements of secondary coils 36 which may be placed on the inside front surface of the housing 30 (as at A) or, alternatively, on the outside front surface (as at B). The inside mounting (A) is possible with non-metallic housings such as plastic or composite, and avoids the need to seal the patterned grooves with silicon or the like. In either case, the secondary coils 36 reside flat against the housing 30 by seating them in grooves that conform to the surface profile.

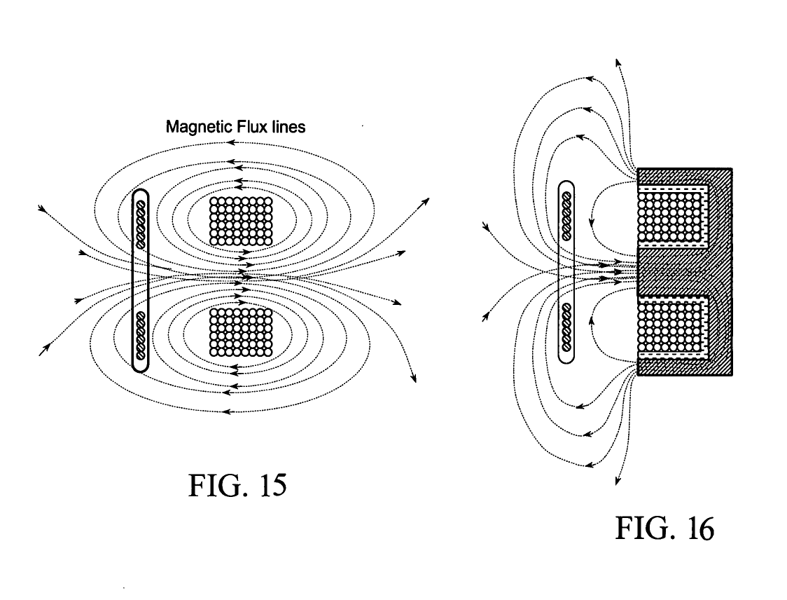

FIG. 15 illustrates the magnetic flux coupling path imparted by the present device, and FIG. 16 illustrate the leakage flux paths imparted by the present device. One of the biggest challenges in medical electronics is controlling electromagnetic interference (EMI) while maintaining the low leakage currents necessary for maximum power transmission. In most electronic devices, EMI is controlled in a known manner by integrating filters, such as Y-capacitor-type filters, to protect against common-mode interference. However, since common-mode interference occurs primarily because of parasitic coupling paths, it is important to keep such paths to a minimum in the design. Leakage flux has the effect of adding inductance that produces a voltage drop when current is present. Leakage can be controlled by the shape of the core and by the arrangement of the windings. In the present design, the core is as compact as possible and the windings close together in order to minimize leakage flux. It also helps to reduce leakage and EMI if a physician ensure that the primary charging unit 4 is optimally aligned with the implanted secondary recharging unit 6 during use. This requires placement of the primary charging unit 4 on the human body as close as possible to the secondary unit 6 for efficient power transmission. For this purpose, the present invention may include a belt or vest that is worn by the patient and that suspends the primary charging unit 4 at the correct position on the body. Given an array of pockets, the primary unit 4 can be disposed at various points on the belt/vest.

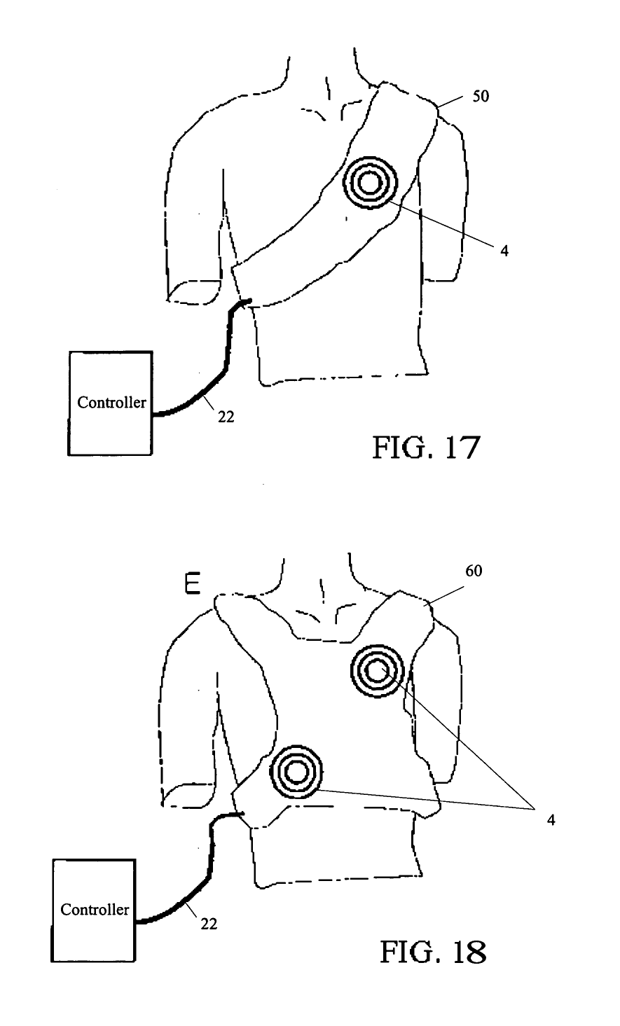

FIG. 17 is a perspective drawing of one embodiment of a shoulder strap 50 designed to be worn to suspend the primary charging unit 4 at the correct position on the body. Again, given one or more (an array) of pockets along the inside of shoulder strap 50, the primary unit 4 can be disposed at any one of various points on the strap 50, thereby ensuring pinpoint positioning of the primary charging unit 4 relative to the secondary unit 6.

FIG. 18 is a perspective drawing of another embodiment which is a vest 60, again designed to be worn to suspend one or more primary charging units 4 at the correct positions on the body. Again, given one or more (an array) of pockets along the inside of vest strap 50, a plurality of primary units 4 can be disposed at a plurality of points on the vest 60, thereby ensuring pinpoint positioning.

It should now be apparent that the foregoing transcutaneous power transmission system for use in an implantable medical device offers is extremely small and compact and minimizes surgery and subsequent treatments. The specific configuration of the primary unit 4 and secondary unit 6 optimizes the transcutaneous magnetic coupling to minimize charging time. The system can be utilized for various implantable medical devices that requires electrical power, such as an artificial heart, a pacemaker, an implantable cardiverter defibrillator, a neurostimulator, a GI stimulator, an implantable drug infusion pump, a bone growth stimulation device, and many other devices. Sufficient electric power can be transmitted to the medical device repeatedly without having to take the implanted medical device out of the human body. Further, since charging is more convenient the size of the battery 44 can be reduced, thereby reducing the overall size of the implanted medical device. Since the secondary coil(s) 36 can be formed in a variety of shapes in or on the housing 30, it is easy to design medical devices that conform to the inside of a living body.

Having now set forth the preferred embodiments and certain modifications of the concepts underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth in the appended claims.

1. A transcutaneous recharging system, comprising:
a primary recharging unit including,
a toroidal housing,
a ferrite core seated in said housing,
a bobbin seated in said ferrite core,
a charging coil wound about said bobbin for producing magnetic flux in a coaxial direction,
a composite cover sealing the charging coil, bobbin, and ferrite core inside the toroid-shaped housing; and
an implantable electronic medical device, including,
a housing,
circuitry enclosed within said housing,
a rechargeable battery enclosed in said housing for powering said circuitry; and
a secondary recharging unit integrally formed in said housing, said secondary recharging unit comprising a protective layer, and at least one single-layer helical coil inlayed in said housing in advance of said protective layer and sealed therein so as not to disrupt a surface profile of said housing, said secondary coil for producing magnetic flux in a coaxial direction.

2. The transcutaneous recharging system according to claim 1, wherein said secondary housing is defined by a circular recess, and the secondary side coil is contained within the circular recess.

3. The transcutaneous recharging system according to claim 2, wherein said protective layer is a ferromagnetic polymer film covering at least the circular recess.

4. The transcutaneous recharging system according to claim 2, wherein said circular recess is filled with an isolation layer sealant encapsulating said secondary side coil.

5. The transcutaneous recharging system according to claim 4, wherein said isolation layer sealant is silicon sealant for a hermetic seal.

6. The transcutaneous recharging system according to claim 1, further comprising a pair of flux sensors, inclusive of a first flux sensor in said primary recharging unit and positioned along an axis of said ferrite core, and a second flux sensor in said secondary recharging unit.

7. The transcutaneous recharging system according to claim 1, wherein the isolation composite cover of said primary recharging unit has a smaller diameter and protrudes outward from said toroidal housing to serve as a skin depressant to maximize magnetic coupling between the primary and secondary recharging units.

8. The transcutaneous recharging system according to claim 1, wherein the secondary side coils and isolation layer are integrally formed as a flex printed circuit board by laminating the coils between opposing polymer sheets, said sheets serving as isolation layer.

9. The transcutaneous recharging system according to claim 8, wherein said secondary housing is defined by a circular recess, and the flex printed circuit board is inlayed into a recess on the secondary charging unit housing in a flush configuration.

10. A transcutaneous recharging system, comprising:
a primary recharging unit including,
a toroidal housing,
a ferrite core seated in said housing,
a bobbin seated in said ferrite core,
a primary charging coil wound about said bobbin for producing magnetic flux in a coaxial direction,
a composite cover sealing the charging coil, bobbin, and ferrite core inside the toroid-shaped housing; and
an implantable electronic medical device, including,
a housing having a surface defined by at least one helical groove,
circuitry enclosed within said housing,
a rechargeable battery enclosed in said housing for powering said circuitry, and
at least one single-layer helical secondary coil inlayed in the groove of said housing and sealed therein by sealer so as not to disrupt the surface profile of said housing, said secondary coil for producing magnetic flux in a coaxial direction.

11. The transcutaneous recharging system according to claim 10, wherein said helical groove is filled with an isolation layer sealant encapsulating said secondary side coil.

12. The transcutaneous recharging system according to claim 11, wherein said isolation layer sealant is silicon sealant for a hermetic seal.

13. The transcutaneous recharging system according to claim 10, further comprising a pair of flux sensors, inclusive of a first flux sensor in said primary recharging unit and positioned along an axis of said ferrite core, and a second flux sensor in said secondary recharging unit.

14. The transcutaneous recharging system according to claim 10, wherein the isolation composite cover of said primary recharging unit comprises a smaller diameter than and protrudes outward from said toroidal housing to serve as a skin depressant, thereby maximizing magnetic coupling between the primary and secondary recharging units.

15. The transcutaneous recharging system according to claim 10, wherein the at least one helical groove comprises a plurality of helical grooves and said at least one single-layer helical secondary coil comprises a corresponding plurality of single-layer helical secondary coils.

16. The transcutaneous recharging system according to claim 10, wherein the at least one helical groove comprises a convex helical groove conforming to an outer surface contour of said housing.

17. The transcutaneous recharging system according to claim 10, wherein the at least one helical groove comprises a concave helical groove conforming to an outer surface contour of said housing.

18. An apparatus for providing power to an implantable medical device in a living body, comprising:
a primary coil external to said living body, said primary coil being wound concentrically about a first axis and connected to an external power source for producing magnetic flux in an axial direction; and
a flat flexible secondary coil formed by direct printing onto a housing of an implantable medical device internal to a living body, said flat secondary coil being printed as a single-layer conductor wound concentrically about a second axis for receiving power in the form of magnetic flux from said primary coil when said first and second axes are substantially aligned and providing the received power to said implantable medical device.

19. An apparatus for providing power to an implantable medical device, comprising:
a primary coil wound concentrically about a first axis and connected to an external power source for producing magnetic flux; and
a flexible printed circuit board including a flat secondary coil having a single conductor printed concentrically on a polymer substrate about a second axis, said flexible printed circuit board being attached across a housing of said implantable medical device for receiving power in the form of magnetic flux from said primary coil and providing the received power to said implantable medical device.

20. The apparatus of claim 19, wherein said secondary coil is printed directly as a helix on said polymer substrate.

21. The apparatus of claim 19, wherein said polymer substrate is attached directly to the housing of said implantable medical device.

23. The apparatus of claim 21, wherein said polymer substrate is sealed within said recess by silicon sealant.

24. A transcutaneous recharging system, comprising:
a primary recharging unit including,
a toroidal housing having a front surface and a smaller back surface,
a ferrite core seated in said housing,
a bobbin seated in said ferrite core,
a primary charging coil wound about said bobbin for producing magnetic flux in a coaxial direction,
a composite cover sealing the charging coil, bobbin, and ferrite core inside the toroid-shaped housing, said composite cover protruding beyond a plane of said front toroidal housing surface to function as a skin depressant during use to maximize a magnetic coupling of said primary recharging unit; and
an implantable electronic medical device, including,
a housing having a surface defined by at least one helical groove,
circuitry enclosed within said housing,
a rechargeable battery enclosed in said housing for powering said circuitry, and
at least one single-layer helical secondary coil inlayed in the groove of said housing and sealed therein by sealer so as not to disrupt the surface profile of said housing, said secondary coil for producing magnetic flux in a coaxial direction.

25. The apparatus of claim 24, further comprising a shoulder strap for wearing by a patient, said shoulder strap including at least one pocket for suspending said primary charging unit at a predetermined position on said patient's body.

26. The apparatus of claim 24, further comprising a vest for wearing by a patient, said vest including at least one pocket for suspending said primary charging unit at a predetermined position on said patient's body.
